(19) United States
(12) Patent Application Publication
(10) Pub. No.: US 2001/0029354 A1
(43) Pub. Date: Oct. 11, 2001

(54) Method for filing a syringe

(75) Inventors: Alexander Rolle, Schillsdorf (DE); Karl-Heinz Pipprich, Padenstedt (DE)

Correspondence Address: Thomas C. Potani, Esq., Cohen, Potani, Lieberman & Pavane, Suite 1210, 551 Fifth Avenue, New York, NY, 10176, US

(73) Assignee: Transcoject Gesellschaft fur medizinische Gerate mbH & Co. KG

(21) Appl. No.: 09/782,117

(22) Filed: Feb. 13, 2001

(30) Foreign Application Priority Data
Feb. 14, 2000 (DE) 100 06 474.4

(51) Int. Cl. A61M 5/00

(52) U.S. Cl. 604/218; 604/232

(57) ABSTRACT

For filling a syringe firstly the syringe cylinder is filled with a fluid or a pasty substance, whereafter the syringe piston is introduced. Via an opening in the base of the syringe piston the air located in the syringe cylinder is removed, whereupon the opening is closed.

### BACKGROUND OF THE INVENTION

The invention relates to a method for filling a syringe according to the features specified in the introductory part of claim 1 or 2.

For simplifying the handling nowadays there are increasingly offered filled syringes, be it for medical purposes (a syringe filled with medicine) or also for other, for example technical purposes (the syringe is e.g. filled with a lubricant or adhesive). This is effected usually in a manner such that the syringe cylinder firstly at its end on the hollow needle connection side is sealing closed by a cap, whereupon it is filled from the other side. After the desired degree of filling has been achieved finally the syringe piston is introduced into the cylinder. Since the syringe piston is sealed with respect to the syringe cylinder there constantly forms an air cushion between the piston base and the surface of the filled material, this having to be removed. With the syringes consisting of plastic this is usually effected in that the syringe cylinder at two opposed sides is impinged with force and is deformed to the extent that on account of the then arising oval cross section the air may escape laterally past the piston.

This bleeding laterally past the piston has considerable disadvantages. On the one hand there exists the danger that the piston seal is damaged by way of the deformation of the syringe cylinder. Furthermore there exists the danger that the syringe cylinder plastically deforms which leads to problems with regard to the sealedness between the piston and the cylinder, as well as with the sliding of the piston in the cylinder. This method may furthermore not be applied to syringes with which the piston and the piston seal are of two parts, i.e. the piston seal is manufactured of a high-elastic material, since then the seal leads to a cylinder deformaton. Exactly this type of syringe would however be particulary suitable for prefilling since with regard to the sealedness and handling when delivering the filled product it has considerable advantages. Finally it is to be mentioned that the previously mentioned filling method also entails hygenic problems since as a rule it cannot be avoided that on bleeding a part of the filled product situated in the cylinder is entrained and settles on the other side of the piston within the syringe cylinder or on the piston shank.

### BACKROUND OF THE INVENTION

Against this background it is the object of the invention to provide a method for filling syringes with which the previously mentioned disadvantages are avoided. Furthermore there is to be provided a syringe with which the method according to the invention may be carried out. The part of the object with regard to the method is achieved by the features in claim 1 or alternatively claim 2.

The part of the object with regard to the device is achieved by the features specified in claim 3 or claim 12. Advantageous formations of the invention are specified in the dependent claims as well as in the subsequent description.

The method according to the invention, instead of the lateral bleeding past the piston up to now, envisages a bleeding through an opening in the piston base which after the bleeding is sealingly closed. Alternatively also the filling may be effected itself through this opening, for example via a tube which from the proximal side is led through the piston shank up to the opening.

The method according to the invention has the advantage that by way of the filling and bleeding procedure there is exerted no loading on the piston seal. Furthermore pistons with a piston seal designed as one piece as well as such with a separately designed piston seal, for example in the form of an O-ring, may be applied. Since a deformation of the syringe cylinder is no longer required, there is given a considerably improved functional safety of the filled syringe. In particular with the use of a tube reaching up to the opening it may be achieved that on bleeding, should the occasion arise the entrained filled product no longer settles in the region of the piston shank. This is particularly advantageous for the medical application, since thus the highest demands on hygiene may be met.

The method according to the invention according to claim 1 with which the bleeding is effected after introducing the piston into the syringe cylinder, may be carried out largely with present installations according to the state of the art, whilst the method according to the invention according to claim 2 with which the filling procedure is effected through the opening in the piston base will require large modifications of the installation. Common to both methods is that there are applied syringes specially designed for this, in particular with a modified syringe base. The minimum demand with this is an opening in the piston base through which the air located in the part of the cylinder space sealed by the piston (or the gas or gas mixture located here) may be removed. This opening must after the bleeding or filling procedure be sealingly closed which is either effected by a closure plug which is incorporated sealingly into the opening with a positive and/or non-positive fit, or however closed in another manner. According to the invention this opening may also be welded, adhesed, filled-over or closed in another manner.

Whilst with syringes according to the state of the art it is usual on the essentially plate-shaped piston to form a profiled shank with a proximal-side contact surface for the thumb, for the design according to the invention it is advantageous when on the piston base there connects a tubular shank so that the opening situated in the piston base by way of the piston shank is conductingly connected to the proximal end of the piston. Via this tubular shank one may bleed and fill, be it directly or by way of introduction of a tube. Furthermore the closure plug may be led through this shank up to the opening. In order to form a contact surface for the thumb, as is usual with syringe pistons on the proximal side, usefully at the proximal end of the piston shank there is provided a flange-like rest. A thus formed piston may be manufactured as a one-piece injection moulded part. At the same time the piston may be selectively designed as one piece with the piston seal or also comprise a separate piston seal of a more elastic material.

As a closure body for the opening on the base of the piston, a ball, e.g. a plastic ball may be applied, wherein then the opening in the base of the piston may usefully be designed such that the ball locks in the opening with a positive as well as a non-positive fit in order on the one hand to ensure a secure hold in the opening and on the other side also to produce the sealing effect. The application of a ball as a closure body is particularly favorable with regard to assembly technology, since with the supply of the ball no orientation is to be observed and moreover the separation of such balls is technically not so complicated.

Particularly reliable in the sealing effect is a conical or truncated-cone-shaped closure body which either is exclusively brought sealingly into the opening with a non-positive fit or additionally may further be locked. If a conical closure plug is used which in an orientated manner must be introduced into the correspondingly conically formed opening through the piston shank, it is useful to provide the closure body itself with a shank as well as with a proximal-side rest in a manner such that on pushing the piston into the cylinder the force effort is effected via the rest on the closure body side. By way of this it is achieved that then when the piston exerts a pressure on the fluid or pasty mass located in the cylinder, at the same time also the force on the closure body is increased, by which means the sealing effect is reliably ensured also on exiting the substance through the syringe tip.

It is advantageous when such a conical closure body with a shank and proximal-side rest is used, to dimension these such that also the air located in the piston shank at least largely is displaced.

### BRIEF DESCRIPTION OF THE DRAWINGS

The invention is hereinafter described in more detail by way of embodiemnt examples shown in the Figures. There are shown:

FIG. 1a-d four method steps for filling a first embodiment of a syringe according to the invention according to the method according to the invention,

FIG. 2a-d an alternative embodiment of a syringe according to the invention in the representation according to FIG. 1 and

FIG. 3 in an enlarged representation the distal end of the piston of a further embodiment in a longitudinal section.

### DETAILED DESCRIPTION OF THE INVENTION

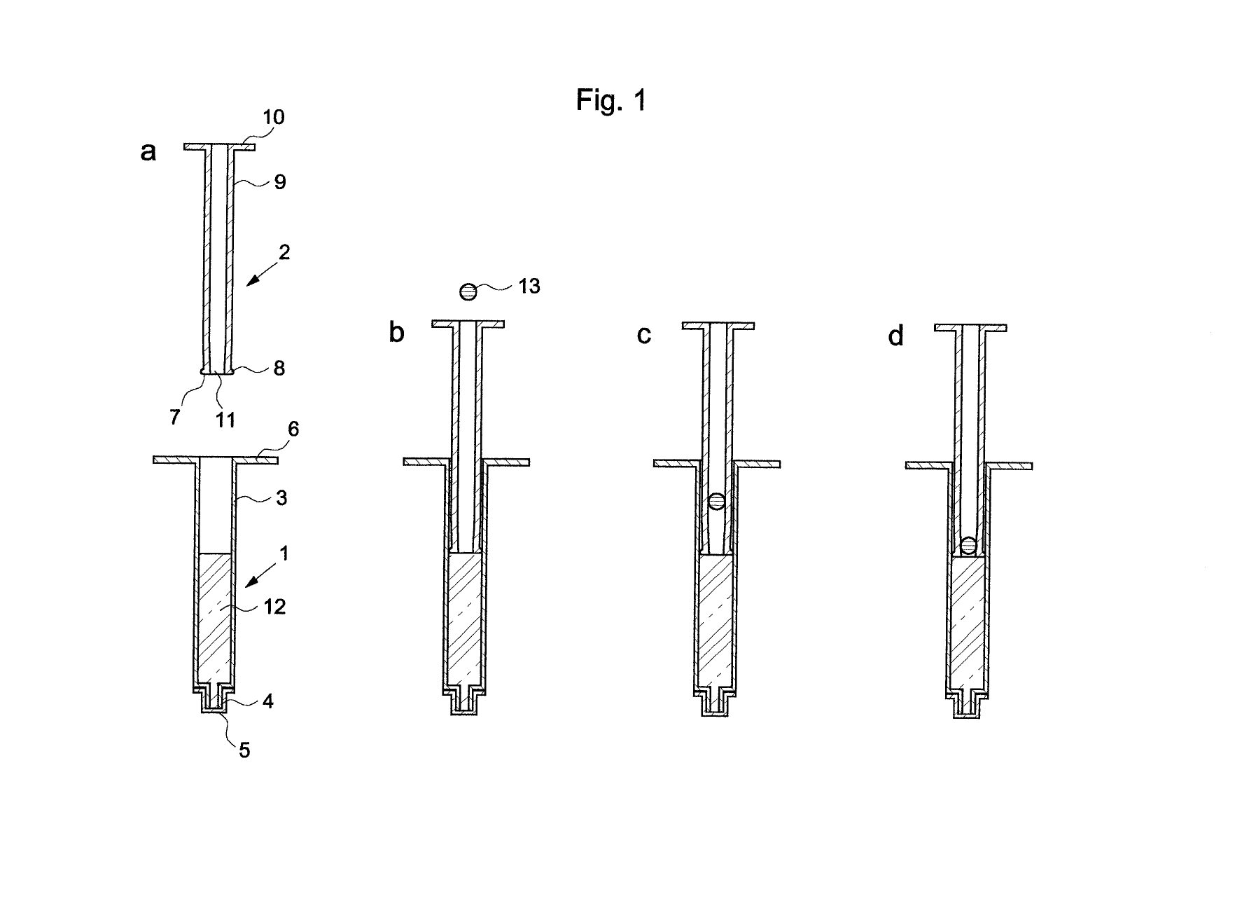

The syringe represented by way of FIG. 1 consists of a syringe cylinder 1 and of a syringe piston 2. The syringe cylinder 1 comprises a cylindrically tubular part 3 which at the distal end is limited by an end-face wall which blends into a hollow needle connection 4, for example into the cone-shaped Luer connection or the Luer lock connection with a thread locking. The hollow needle connection 4 is sealingly closed by a removable closure cap 5. To the proximal end of the tubular part 3 there connects a flange-like part 6 which forms a finger rest.

The syringe piston 2 consists of a piston base 7 which forms the actual piston and which on the circumferential side comprises a piston seal 8. To the piston base 7 there proximally connects a tubular piston shank 9 which at its proximal end comprises an outwardly directed flange 10.

In the piston base 7 there is provided an opening 11 which is arranged centrally and blends in a flush manner into the inner walling of the piston shhank 9, as this may be clearly seen in FIG. 1a.

The syringe piston 2 is dimensioned such that on introduction into the syringe cylinder 1, it with the piston seal 8 sealingly bears and is displaceable within the tubular part 3.

For filling the syringe with a fluid or pasty substance 12 the syringe cylinder 1 sealingly closed with the cap 5, in the arrangement shown in FIG. 1a, from above, i.e. from the flange-like part 6, is filled and specifically up to the desired level of filling. After this has been effected (FIG. 1a) the syringe piston 2 is introduced into the syringe cylinder 1 and is pushed so far into this until the distal piston end has reached the surface of the filled material 12. With this the air located in the tubular part 3 between the piston base 7 and the surface of the filled material 12 in the tubular part 3 is removed to the top (FIG. 1b) via the opening 11 and the piston shank 9. As soon as this position has been reached, thus the air located between the piston base 7 and the filled material 12 (this may also be any other gas or gas mixture) has been removed, the opening 111 is sealingly closed by a closure body 13 in the form of a ball. For this the ball 13 from the flange-side end of the syringe piston 2 is introduced into the piston shank 9 (FIG. 1b, 1c). Since the diameter of the ball is considerably smaller than the inner diameter of the tubular piston shank 9 the ball 13 reaches up to near the piston base 7. The air which is displaced with this may flow laterally past between the piston shank and the ball. Shortly before reaching the piston base 7 the piston shank 9 tapers on its inner side up to below the ball diameter so that the ball here on account of the piston shank 9 becoming narrower sticks (FIG. 1c). The ball 13 then by way of a tool introduced into the piston shank 9 from the flange-side end is pressed into the closure position shown in FIG. 1d in which it sealing closes the opening 11 with a positive and non-positive fit. For this within the piston base 7 there is provided a type of valve seat for the ball 13 which is selected such that by way of a non-positive fit a circumferential sealing line is achieved, wherein the opening above and below this sealing seat is tapered such that there is given a positive fit which holds the ball 13 in this position. On pressing the ball into the sealing seat this positive fit connection by widening is overcome for a short time so that there arises a type of locking connection. Since this narrowed region is seated above the piston seal 8 the piston seal by way of this temporary widening is not compromised, even when this as with the embodiment shown in FIG. 1 is formed as one piece with the remaining syringe piston 2.

In place of the previously described method the syringe cylinder 1 also after applying the syringe piston 2 may be filled, wherein then the filling is effected by way of a tube which is led through the piston shank 9 up to the opening 11 and via which the filled product 12 is brought in. After removing this tube there is then effected the closing of the opening 11 as is described by way of FIGS. 1b to d.

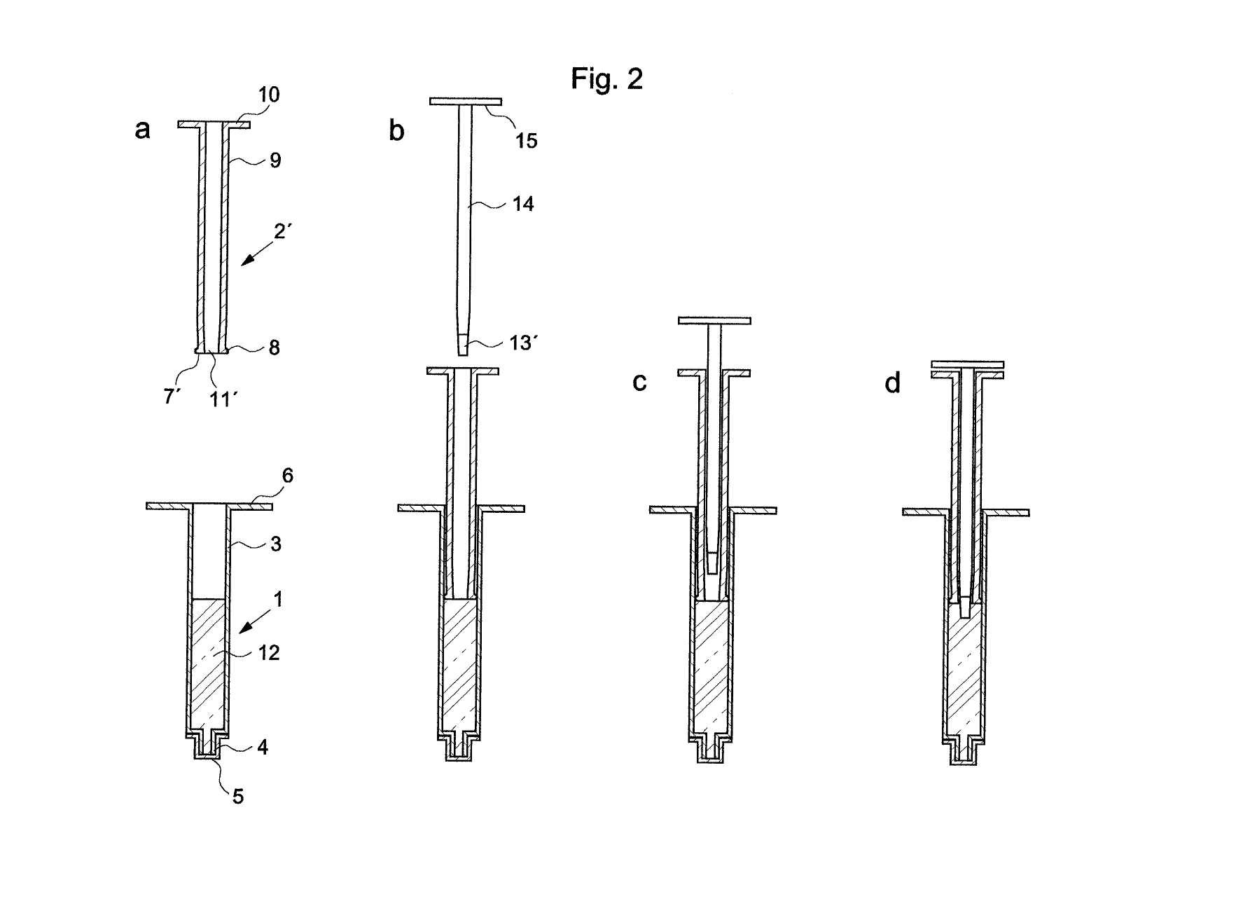

By way of the FIGS. 2a-d the same filling procedure is described, however here the syringe piston 2 differs with respect to the opening 11 as well as the closure body 13.

The syringe piston 2′ represented in FIG. 2 differs from the previosuly described syringe piston 2 essentially by the design of the opening 11′ in the piston base 7′. The opening 11′ is designed conically tapering towards the distal end. Correspondingly conically designed is the closure body 13′ which apart from the actual truncated-cone-shaped closure body part also on the proximal side comprises a shank 14 which is led through the piston shank 9 and projects beyond this on the proximal side. At the proximal end of the shank 14 there is provided a plate-shaped rest 15.

As with the previously described embodiment firstly the syringe cylinder 1 is filled with filling material 12, whereupon the syringe piston 2′ is introduced until the piston base 7′ bears on the surface of the filling material 12. Then the closure body 13′ from the flange-side end of the syringe piston 2 is introduced into the piston shank 9, wherein the closure body 13 and the shank 14 are dimensioned such that they may be led through the piston shank 9 with play. The closure body 13′ reaches its closure position when this is sealingly seated in the opening 11′ as this is represented by way of FIG. 2d. With this basically a non-positive fitting seat of the closure body 13′ in the opening 11′ is sufficient. However additionally between the shank 14 and the inner side of the piston shank 9 there may be provided a latching device, for example by way of a projection in the piston shank 9 with a correspniding indentation in the cylinder 3 which additionally secures this closure position with a positive fit. Furthermore with this embodiment of the closure body 13′ it is additionally secured that in the application case, when thus the syringe piston 2 for delivering the filling material 12 via the hollow needle connection 4 is moved into the syringe cylinder 1, the force effort on the piston side is effected via the rest 15, i.e. the force transmission is compellingly effected via the sealing seat between the closure body 13′ and the opening 11′.

For improving the bleeding effect it is useful to form the piston base 7′ running obliquely to the opening 11 or 11′ so that the air located between the piston base 7′ and the surface of the filling material 12 practically automatically is led towards the opening and a complete as possible bleeding may be effected.

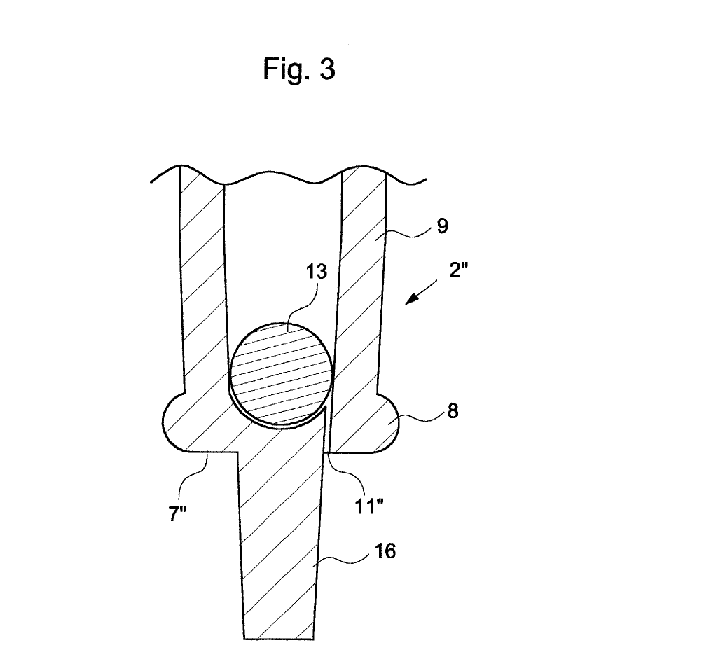

In FIG. 3 there is represented one embodiment variant with which the syringe piston 2″ is designed as a so-called displacing piston, i.e. comprises a distal part 16 which in this design is roughly cylindrical and extends distally from the piston base 7″ and serves when inserting for completely emptying the syringe cylinder 1, i.e. including the contents of the hollow needle connection. Since this part 16 must be arranged centrally with this embodiment variant the opening 11″ in the piston base is arranged off-centre, specifically next to the cylindrical part 16. Otherwise this syringe piston 2″ is designed as described by way of FIG. 1.

It is to be pointed out that piston base within the context of the invention is to be understood as the distal side end-face of the syringe piston. Whilst with larger volume pistons as a rule a plate-like base part is located between the opening and the piston base, with syringe pistons of a smaller diameter the piston base may also be formed by the annularly thickened distal end of the syringe piston between the opening and the piston seal, which may continuously blend into the piston shank as is also represented with the above described embodiment examples.

### LIST OF REFERENCE NUMERALS

1—syringe cylinder

2—syringe piston

2′—syringe piston in FIG. 2

2″—syringe piston in FIG. 3

3—tubular part of the syringe cylinder

4—hollow needle connection of the syringe cylinder

5—closure cap

6—flange-like part of the syringe cylinder

7—piston base

7′—piston base in FIG. 2

7″—piston base in FIG. 3

8—piston seal

9—piston shank

10—piston flange

11—opening in the piston base

11′—opening in the piston base in FIG. 2

11″—opening in the piston base in FIG. 3

12—filling material

13—closure body

13′—closure body in FIG. 2

14—shank of the closure body

15—rest of the closure body

16—cylindrical part in FIG. 3

1. A method for filling a syringe with which a syringe cylinder closed at one end is filled with a fluid or pasty substance, whereupon a syringe piston is introduced into the syringe cylinder and gas or a gas mixture located in the part of the syringe cylinder closed by the piston is removed, wherein the removing of the gas or gas mixture is effected via an opening provided in the piston base, which is thereafter closed.

2. A method for filling a syringe with a fluid or pasty substance, wherein into a syringe cylinder closed at one end there is introduced a syringe piston, whereupon the part of the syringe cylinder closed by the piston is filled through an opening in the piston base and subsequently the opening is closed.

3. A syringe, in particular a medical syringe, with a syringe cylinder and a syringe piston displaceably guided therein, wherein in the piston base there is provided a closable opening.

4. A syringe according to claim 3, wherein the piston base comprises a tubular piston shank connecting thereto on the proximal side, wherein the piston shank is conductingly connected to the opening in the piston base.

5. A syringe according to claim 3 or 4, wherein a closure body is provided which is sealingly pressable into the opening.

6. A syringe according to one of the preceding claims, wherein the closure body is formed truncated cone shaped.

7. A syringe according to one of the preceding claims, wherein the closure body is a ball which may be applied with a non-positive and positive fit into the opening of the piston base from the proximal direction.

8. A syringe according to one of the preceding claims, wherein to the piston shank on the proximal side there connects a flange-shaped rest.

9. A syringe according to one of the preceding, wherein the syringe piston with its piston base, with the piston shank and its rest is designed as one piece as an injection moulded part.

10. A syringe according to one of the preceding claims, wherein the closure body is provided with a shank with a proximal-side rest.

11. A syringe according to one of the preceding claims, wherein the closure body and its shank are dimensioned such that the free space formed by the opening and the shank, in the syringe piston, may be almost completely filled out.

12. A piston for a medical syringe, wherein in the piston base there is provided a closable opening.
